(19) United States
(12) Patent Application Publication
(10) Pub. No.: US 2022/0087920 A1
(43) Pub. Date: Mar. 24, 2022

(54) COMPOSITION COMPRISING A MODIFIED PHOTO-DIMERIZABLE POLYMER AND AN ALKALINE AGENT AND/OR AN AMINO ALKOXYSILANE DERIVATIVE AND TREATMENT METHOD IMPLEMENTING THE COMPOSITION

(71) Applicant: L'OREAL, Paris (FR)

(72) Inventors: Frédérick WOODLAND, SAINT-OUEN (FR); Eric PARRIS, SAINT-OUEN (FR); Gabin VIC, SAINT-OUEN (FR)

(21) Appl. No.: 17/312,646

(22) PCT Filed: Dec. 17, 2019
(86) PCT No.: PCT/EP2019/085605

§ 371 Date: Jun. 10, 2021

(30) Foreign Application Priority Data
Dec. 21, 2018 (FR) 1873911

(51) Int. Cl. A61K 8/81; A61K 8/31; A61Q 5/00; A61K 8/58; A61K 8/44

(52) U.S. Cl. CPC A61K 8/8117; A61K 8/31; A61K 8/44; A61K 8/585; A61Q 5/00

(57) ABSTRACT

The present invention concerns a composition comprising: —at least one photo-crosslinkable polymer comprising at least one photo-dimerizable pendant group; —one or more alkaline agent(s) and/or amino alkoxysilane derivative(s), the alkaline agent(s) and/or amino alkoxysilane derivative(s) being present in the composition in an amount ranging from 0.01 to 10% by weight relative to the total weight of the composition, the pH of the composition being greater than or equal to 7.

The present invention relates to a composition, notably a cosmetic composition, notably a hair composition, comprising at least one modified photo-crosslinkable polymer and an alkaline agent and/or an amino alkoxysilane derivative in a particular content. Such a composition can produce a coating that is resistant to washing, the performance of which is stable over time.

It is known for the person skilled in the art to use photo-crosslinkable materials such as materials from vinyl monomers, and notably (meth)acrylate monomers.

Accordingly, U.S. Pat. No. 5,300,285 describes a process for waving hair and a composition for performing this process, and notably a silicone-based neutralizing composition, including a silicone with a vinyl function, a free-radical photoinitiator and a solvent. This composition acts as a substitute for neutralizing solutions based on hydrogen peroxide when it is applied to hair after a process of permanent deformation of the hair and photo-crosslinking of the composition. This composition has the advantage of functioning simultaneously as a crosslinking agent for keratin, while giving hair advantages such as conditioning and a soft feel. Photo-crosslinking of the composition is triggered by the photoinitiator (generally an acetophenone derivative), which releases a radical when exposed to radiation in the UVA domain (350-385 nm).

Unfortunately, photo-polymerization of the silicone with a vinyl function in the presence of a photoinitiator leads to irreversible silicone photo-crosslinking with a vinyl function. In otherwords, once it is photo-crosslinked, the deposit cannot be easily and at any moment removed from the hair.

Document EP1572139 describes a photo-dimerizable composition that can make a deposit on keratin materials, particularly hair. This document teaches that such deposits allow a long-lasting deposit to be obtained that provides durable cosmetic properties over time and are easily removed. The deposits obtained from the crosslinkable compounds described in this document do however have limited persistence. Moreover, these compounds are not deposited uniformly on the hair and the deposit depends greatly on the amount of hair damage. Accordingly, the result is not uniform between the hair's roots and tips.

To obtain a uniform deposit on the entire head of hair, it has already been proposed to use polymers with photo-dimerizable pendant groups and hydrophobic pendant groups, as described notably in WO 2017/108767. The photo-dimerizable polymer described is, however, sparingly stable over time. The performance and safety of the composition are not necessarily ensured over time.

There is a need to further improve the results obtained in terms of stability.

The present invention therefore relates to providing a composition comprising a photo-crosslinkable polymer that does not present the drawbacks of the compositions of the state of the art. The aim of the invention is notably to develop photo-dimerizable compositions that are stable over time, which allow efficient, uniform, reproducible and long-lasting coating of keratin materials, and notably the hair, which coating withstands washing.

Accordingly, one of the subjects of the present invention is a composition comprising:
at least one photo-crosslinkable polymer including at least one pendant photo-dimerizable group:
one or more alkaline agents and/or amino alkoxysilane derivatives, the alkaline agent(s) and/or amino alkoxysilane derivative(s) being present in the composition in a content ranging from 0.01% to 10% by weight relative to the total weight of the composition, the pH of the composition being greater than or equal to 7.

Another subject of the invention is a process for treating keratin materials comprising the application of said composition and a step of irradiating the composition on the keratin materials to crosslink the composition.

Compositions that are stable over time, which make it possible to obtain uniform coating of the hair that is persistent on washing, are thus obtained.

For the purposes of the present invention and unless otherwise indicated:
an “alkylene chain” represents an acyclic C₁-C₂₀ divalent hydrocarbon chain, particularly a C₁-C₆ chain, more particularly a C₁-C₂ chain when the chain is linear, optionally substituted with one or more groups, which may be identical or different, chosen from i) hydroxyl, ii) (C₁-C₂)alkoxy, iii) (poly)hydroxy(C₂-C₄)alkoxy(di)(C₁-C₂)(alkyl)amino, iv) R^{a}—Z^{a}—C(Z^{b})—Z^{c}—, and v) R^{a}—Z^{a}—S(O)ₜ—Z^{c}— with Z^{a} and Z^{b}, which may be identical or different, representing an oxygen or sulfur atom, or a group NR^{a′}, Z^{c}, representing a bond, an oxygen or sulfur atom, or a group NR^{a}; R^{a}, representing an alkali metal, a hydrogen atom, an alkyl group, or alternatively is absent if another part of the cationic molecule and R^{a′} representing a hydrogen atom or an alkyl group; more particularly, the groups iv) are chosen from carboxylate —C(O)O— or —C(O)OMetal (Metal=alkali metal), carboxyl —C(O)—OH, guanidino H₂H—C(NH₂)—NH—, amidino H₂H—C(NH₂)—, (thio)ureo H₂N—C(O)—NH— and H₂N—C(S)—NH—, aminocarbonyl —C(O)—NRa′₂ or aminothiocarbonyl —C(S)—NRa′₂; carbamoyl Ra′—C(O)—NRa′— or thiocarbamoyl Ra′—C(S)—NRa′— with Ra′, which may be identical or different, representing a hydrogen atom or a (C₁-C₄)alkyl group;
the “aryl” or “heteroaryl” radicals or the aryl or heteroaryl part of a radical may be substituted with at least one substituent borne by a carbon atom, chosen from:
   a C₁-C₁₆ and preferably C₁-C₈ alkyl radical, optionally substituted with one or more radicals chosen from the radicals hydroxyl, C₁-C₂ alkoxy, C₂-C₄ (poly)hydroxyalkoxy, acylamino, amino substituted with two C₁-C₄alkyl radicals, which may be identical or different, optionally bearing at least one hydroxyl group, or the two radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;
   a halogen atom;
   a hydroxyl group;
   a C₁-C₂ alkoxy radical;
   a C₂-C₄ (poly)hydroxyalkoxy radical;
   an amino radical;
   a 5- or 6-membered heterocycloalkyl radical;
   an optionally cationic 5- or 6-membered heteroaryl radical, preferentially imidazolium, optionally substituted with a (C₁-C₄)alkyl radical, preferentially methyl;
   an amino radical substituted with one or two identical or different C₁-C₆ alkyl radicals, optionally bearing at least:
i) one hydroxyl group,
ii) one amino group optionally substituted with one or two optionally substituted C₁-C₃ alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
iii) one quaternary ammonium group —N⁺R′R″R′″, M⁻ for which R′, R″ and R′″, which may be identical or different, represent a hydrogen atom or a C₁-C₄ alkyl group; and M⁻ represents the counterion of the organic or inorganic acid or of the corresponding halide;
iv) or one optionally cationic 5- or 6-membered heteroaryl radical, preferentially imidazolium, optionally substituted with a (C₁-C₄)alkyl radical, preferentially methyl;
   an acylamino radical (—NR—C(O)—R′) in which the R radical is a hydrogen atom or a C₁-C₄ alkyl radical optionally bearing at least one hydroxyl group and the R′ radical is a C₁-C₂ alkyl radical;
   a carbamoyl radical ((R)₂N—C(O)—) in which the R radicals, which may be identical or different, represent a hydrogen atom or a C₁-C₄ alkyl radical optionally bearing at least one hydroxyl group;
   an alkylsulfonylamino radical (R′—S(O)₂—N(R)—) in which the R radical represents a hydrogen atom or a C₁-C₄ alkyl radical optionally bearing at least one hydroxyl group and the R′ radical represents a C₁-C₄ alkyl radical or a phenyl radical; an aminosulfonyl radical ((R)₂N—S(O)₂—) in which the R radicals, which may be identical or different, represent a hydrogen atom or a C₁-C₄ alkyl radical optionally bearing at least one hydroxyl group,
   a carboxyl radical in the acid or salt form (preferably salified with an alkali metal or a substituted or unsubstituted ammonium);
   a cyano group;
   a nitro or nitroso group;
   a polyhaloalkyl group, preferentially the trifluoromethyl group;
the cyclic, cycloalkyl or heterocyclic part of a non-aromatic radical may be substituted with at least one radical chosen from the following groups:
   hydroxyl;
   C₁-C₄ alkoxy or C₂-C₄ (poly)hydroxyalkoxy;
   C₁-C₄ alkyl;
   alkylcarbonylamino (R—C(O)—N(R′)—) in which the R′ radical is a hydrogen atom or a C₁-C₄ alkyl radical optionally carrying at least one hydroxyl group and the R radical is a C₁-C₂ alkyl radical or an amino radical optionally substituted with one or two identical or different C₁-C₄alkyl groups, themselves optionally carrying at least one hydroxyl group, said alkyl radicals possibly forming, with the nitrogen atom to which they are attached, a saturated or unsaturated and optionally substituted 5- to 7-membered heterocycle, optionally comprising at least one other nitrogen or non-nitrogen heteroatom;
   alkylcarbonyloxy (R—C(O)—O—) in which the R radical is a C₁-C₄ alkyl radical or an amino group optionally substituted with one or two identical or different C₁-C₄ alkyl groups, themselves optionally bearing at least one hydroxyl group, where said alkyl radicals may form, with the nitrogen atom to which they are attached, a saturated or unsaturated and optionally substituted 5- to 7-membered heterocycle optionally comprising at least one other nitrogen or non-nitrogen heteroatom;
   alkoxycarbonyl (R-G-C(O)—) in which the R radical is a C₁-C₄ alkoxy radical, G is an oxygen atom or an amino group optionally substituted with a C₁-C₄ alkyl group optionally bearing at least one hydroxyl group, where said alkyl radical may form with the nitrogen atom to which they are attached a saturated or unsaturated, optionally substituted 5- to 7-membered heterocycle, optionally comprising at least one other nitrogen or non-nitrogen heteroatom;
a cyclic, cycloalkyl or heterocyclic radical, or a non-aromatic part of an aryl or heteroaryl radical, which may also be substituted with one or more oxo groups;
a cycloalkyl radical is a mono- or bicyclic, hydrocarbon-based radical comprising 3 to 10 carbon atoms, preferably 4 to 7 carbon atoms, such as cyclopentyl or cyclohexyl;
a hydrocarbon-based chain is unsaturated when it includes one or more double bonds and/or one or more triple bonds;
an “aryl” radical represents a monocyclic or fused or non-fused polycyclic carbon-based group comprising from 6 to 22 carbon atoms, and in which at least one ring is aromatic; preferentially, the aryl radical is a phenyl, biphenyl, naphthyl, indenyl, anthracenyl or tetrahydronaphthyl;
a “heteroaryl radical” represents an optionally cationic, 5- to 22-membered, monocyclic or fused or non-fused polycyclic group, comprising from 1 to 6 heteroatoms chosen from nitrogen, oxygen, sulfur and selenium, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthyl and the ammonium salt thereof;
a “heterocyclic radical” is a 5- to 22-membered monocyclic or fused or non-fused polycyclic radical which may contain one or two unsaturations but is non-aromatic, comprising from 1 to 6 heteroatoms chosen from nitrogen, oxygen, sulfur and selenium atoms;
a “heterocycloalkyl radical” is a heterocyclic radical comprising at least one saturated ring;
a “cationic heteroaryl radical” is a heteroaryl group as defined above that includes an endocyclic or exocyclic quaternized cationic group,
when the cationic charge is endocyclic, it is included in the electron delocalization via the mesomeric effect; for example, it is a pyridinium, imidazolium or indolinium group:

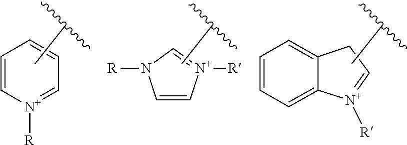

with R and R′ being a heteroaryl substituent as defined above and particularly a (hydroxy)(C₁-C₈)alkyl group such as methyl;
when the cationic charge is exocyclic, it is, for example, an ammonium or phosphonium R⁺ substituent, such as trimethylammonium, which is outside the heteroaryl, such as pyridinyl, indolyl, imidazolyl or naphthalimidyl, in question:

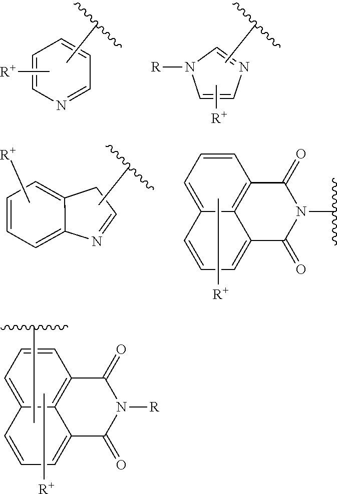

with R a heteroaryl substituent as defined above and R⁺ an ammonium RₐR_{b}R_{c}N⁺—, phosphonium RₐR_{b}R_{c}P⁺— or ammonium RₐR_{b}R_{c}N⁺—(C₁-C₆)alkylamino group with Rₐ, R_{b} and R_{c}, which may be identical or different, representing a hydrogen atom or a (C₁-C₈)alkyl group such as methyl;
a “cationic aryl carrying an exocyclic charge” means an aryl ring whose quaternized cationic group is outside said ring; it is notably an ammonium or phosphonium R⁺ substituent, such as trimethylammonium, which is outside the aryl, such as phenyl or naphthyl:

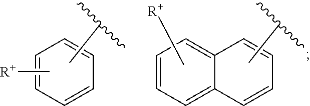

an “alkyl radical” is a linear or branched C₁-C₂₀ and preferably C₁-C₈ hydrocarbon-based radical;
an “alkenylene radical” is an unsaturated hydrocarbon-based divalent radical as defined previously, which may contain from 1 to 4 conjugated or unconjugated double bonds —C═C—; the alkenylene group particularly contains 1 or 2 unsaturations;
the term “optionally substituted” applied to the alkyl radical means that said alkyl radical may be substituted with one or more radicals chosen from the following radicals: i) hydroxyl, ii) C₁-C₄ alkoxy, iii) acylamino, iv) amino optionally substituted with one or two identical or different C₁-C₄ alkyl radicals, where said alkyl radicals may form with the nitrogen atom that bears them a 5- to 7-membered heterocycle, optionally comprising one other nitrogen or non-nitrogen heteroatom; v) or a quaternary ammonium group —N⁺R′R″R′″, M⁻ for which R′, R″ and R′″, which may be identical or different, represent a hydrogen atom or a C₁-C₄ alkyl group, or else —N⁺R′R″R′″ forms a heteroaryl such as imidazolium optionally substituted with a C₁-C₄ alkyl group, and M-represents the counterion of the corresponding organic acid, inorganic acid or halide;
an “alkoxy radical” is an alkyloxy radical for which the alkyl radical is a linear or branched C₁-C₁₆ and preferentially C₁-C₈ hydrocarbon-based radical; when the alkoxy group is optionally substituted, this implies that the alkyl group is optionally substituted as defined above;
“organic or inorganic acid salt” more particularly means the salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
“Anionic counterion” is intended to mean an anion or an anionic group derived from an organic or inorganic acid salt which counterbalances the cationic charge of the colorant; more particularly, the anionic counterion is chosen from: i) halides such as chloride or bromide; ii) nitrates; iii) sulfonates, including C₁-C₆ alkylsulfonates: Alk-S(O)₂O⁻ such as methanesulfonate or mesylate and ethanesulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O⁻ such as methyl sulfate and ethyl sulfate; x) aryl sulfates: Ar—O—S(O)O⁻ such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻, xiii) phosphates O═P(OH)₂—O⁻, O═P(O⁻)₂—OH O═P(O⁻)₃, HO—[P(O)(O⁻)]_{w}—P(O)(O⁻)₂ with w being an integer; xiv) acetate; xv) triflate; and xvi) borates such as tetrafluoroborate, xvii) disulfate (O═)₂S(O⁻)₂ or SO₄²⁻ and monosulfate HSO₄⁻;
the anionic counterion, derived from an organic or inorganic acid salt, ensures the electrical neutrality of the molecule; thus, it is understood that when the anion comprises several anionic charges, then the same anion may serve for the electrical neutrality of several cationic groups in the same molecule or else may serve for the electrical neutrality of several molecules; for example, polymers that comprise two cationic species may contain either two “singly charged” anionic counterions or one “doubly charged” anionic counterion such as (O═)₂S(O⁻)₂ or O═P(O⁻)₂—OH;
moreover, the addition salts that may be used in the context of the invention are notably chosen from addition salts with a cosmetically acceptable base such as alkaline agents as defined below, for instance alkali metal hydroxides, such as sodium hydroxide or potassium hydroxide, aqueous ammonia, amines or alkanolamines;
the expression “at least one” is equivalent to “one or more”; and
the expression “inclusive” for a range of concentrations means that the limits of the range are included in the defined range.

For the purposes of the present invention, the term “photo-dimerizable group” means a chemical group that leads to photo-dimerization reactions under irradiation. For the purposes of the invention, photo-dimerization is a chemical reaction between two double bonds (of 2+2 type) or two pairs of double bonds (of 4+4 type), and more particularly between two double bonds (of 2+2 type).

The case of a reaction between two double bonds may be represented schematically in the following manner:

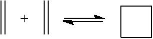

These photo-dimerization reactions are defined in the book Advanced Organic Chemistry, J. Marck, 4th edition, Wiley Interscience, N Y 1992, page 855.

Thus, the double bond, when it is photo-stimulated, generally when it is subjected to specific UV radiation, proves to be capable of reacting with another double bond by cyclization.

According to the present invention, the double bond is said to be activated, i.e. it is spontaneously photo-dimerizable, without requiring the presence of a photoinitiator or a chemical initiator.

This double bond is generally activated by the presence of an electron-withdrawing substituent in the alpha position of this photo-dimerizable double bond.

As electron-withdrawing substituent mention may be made of aromatic rings such as the phenyl group optionally substituted with one or more halogen atoms, or electron-withdrawing groups such as NO₂, CN, R′—Y—C(Y′)—, R′—C(Y′)—Y—, R′—Y—C(Y′)—Y—, R′—Y—S(O)₂—, —S(O)₂—Y—R′, where R′ represents a hydrogen atom or a (C₁-C₄)alkyl group optionally substituted with one or more halogen atoms, where Y and Y′, which may be identical or different, represent an oxygen or sulfur atom or NR″ where R″ represents a hydrogen atom or a (C₁-C₆)alkyl group.

The process according to the invention comprises a step a) of applying a composition comprising a polymer including at least one photo-dimerizable pendant group.

Preferably, the photo-dimerizable pendant groups that may be used according to the invention are chosen from monovalent radicals having the following formulae (I) and (II):

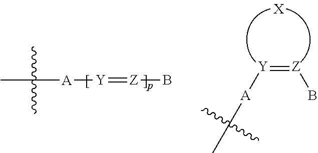

and the geometric isomers thereof,
in which formulae (I) and (II):
Y and Z independently denote a nitrogen atom or a group C(R) where R represents a hydrogen atom or a (C₁-C₄)alkyl group such as methyl;
A represents one bond or a divalent group chosen from (C₁-C₈)alkylene, arylene, heteroarylene, cycloalkylene, heterocycloalkylene, (thio)carbonyl, (C₂-C₈)alkenylene radicals and combinations thereof;
B represents a monovalent group chosen from (C₁-C₈)alkyl radicals, aryl, optionally cationic heteroaryl, cycloalkyl, optionally cationic heterocycloalkyl, (thio)carbonyl, (C₂-C₈)alkenyl radicals and combinations thereof;
X represents a divalent group chosen from (C₂-C₈)alkylene, arylene, heteroarylene, cycloalkylene, heterocycloalkylene, (thio)carbonyl, (C₂-C₈)alkenylene radicals and combinations thereof;
p represents an integer between 1 and 5 inclusive, more particularly between 1 and 3, preferably p is 1;

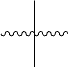

represents the bond that connects the part of the monovalent radical to the rest of the molecule;
and
where each of the groups cited can optionally be substituted with one or more halogen atoms or groups chosen from (C₁-C₆)alkyl, hydroxy, amino, (di)(C₁-C₆)alkylamino, phenyl, carboxy, (C₁-C₆)alkoxy, (C₁-C₆)alkoxy(thio)carbonyl, hydrogen(thio)-carbonyl, sulfonato R—O—S(O)₂— or R—S(O)₂—O—, amide RR′N—C(O)— or R—C(O)—N(R′)— or acyl R—C(O)—, ammonium RR′R″N⁺— groups, where R, R′, and R″, which may be identical or different, represent a hydrogen atom or a (C₁-C₄)alkyl group.

The pendant dimerizable groups according to the invention are notably those cited in U.S. Pat. No. 2,811,510, EP 0 313 220, EP 0 313 221, EP 092 901, GB 2 030 575 and GB 2 076 826, and in the articles “Chemical Review Vol 83, 5 1983, p 507” “Polym, Paint Colour Journal 1988, 178, p 209” and “Current Trends in Polymer Photochemistry, Ellis Morwood edition, NY, 1995”.

As examples, photo-dimerizable pendant groups chosen from monovalent radicals from the following compounds may more particularly be cited:
stilbene,
styrylpyridinium (stilbazolium) having formula and the geometric isomers thereof:

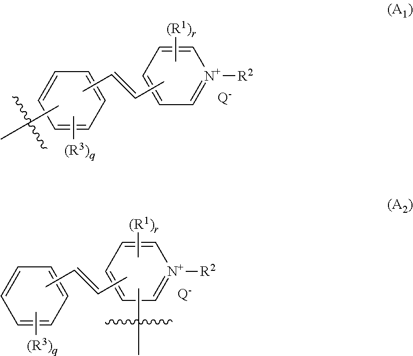

where:
R¹ and R³, which may be identical or different, represent a halogen atom or a (C₁-C₆)alkyl group; or else two contiguous R¹ or R³ groups together form, with the carbon atoms that bear it, a benzo group;
R² represents a hydrogen atom, a (C₁-C₆)alkyl group optionally substituted with one or more halogen atoms such as chlorine or hydroxy, preferably R² represents a (C₁-C₆)alkyl group such as methyl, ethyl, propyl;
q and r represent an integer between 0 and 4 inclusive; and
Q⁻ represents an anionic counterion preferably chosen from halide ions such as chlorides, bromides, iodides, perchlorates, tetrafluoroborates, methylsulfate, phosphates, sulfates, methanesulfonates, p-toluenesulfonate;

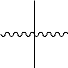

represents the bond that connects the part of the monovalent radical to the rest of the molecule, it being understood that the pendant group A₂ may be connected to the rest of the molecule via R²;

preferably the

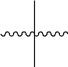

bond is found on the phenyl in the para position relative to the styryl group on A₁ or connected to the rest of the molecule via R² on A₂; preferentially the styryl group of A₁ and A₂ is found para to the pyridinium group;
styrylazolium having formula and the geometric isomers thereof:

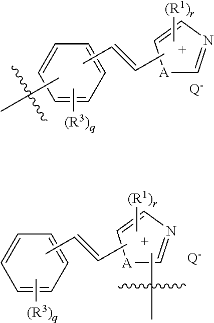

where:
A represents a sulfur atom, an oxygen atom, or an NR² or C(R²)₂ group; and

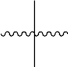

Q⁻, r, q, R¹, R², and R³ are as defined previously, preferably the

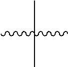

bond is found on the phenyl in the para position relative to the stryryl group,
styrylpyrazine,
chalcone,
(thio)cinnamate and (thio)cinnamamide,
maleimide,
(thio)coumarin,
thymine,
uracil,
butadiene
anthracene,
pyridone,
pyrrolizinone,
acridizinium salts,
furanone,
phenylbenzoxazole, and
derivatives thereof.

According to a particular embodiment, the photo-dimerizable pendant group(s) of the invention are chosen from:

a) photo-dimerizable group(s) bearing a stilbazolium function having formula (Ia) or (Ib), and the geometrical isomers thereof:

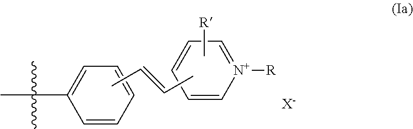

in which:
R represents a hydrogen atom, or a C₁-C₄ alkyl or C₁-C₄ hydroxyalkyl group,
R′ represents a hydrogen atom or a C₁-C₄ alkyl group, and
X⁻ denotes an anionic counterion chosen from halide ions such as chlorides, bromides, iodides, perchlorates, tetrafluoroborates, methyl sulfate, phosphates, sulfates, methanesulfonates, p-toluenesulfonate; preferably the styryl group is found para to the pyridinium group and/or para to the

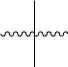

bond;

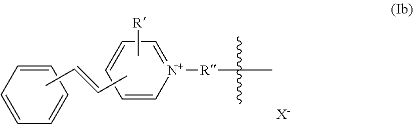

in which
R″ denotes a divalent alkylene radical having from 2 to 8 carbon atoms,
R′ represents a hydrogen atom or a C₁-C₄ alkyl group, and
X⁻ has the same meaning as that described for the preceding formula (Ia);
where

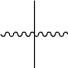

has the same meaning as previously;

preferably the styryl group is found para to the pyridinium group;

or

b) photo-dimerizable groups bearing a styrylazolium function having formula (IIa):

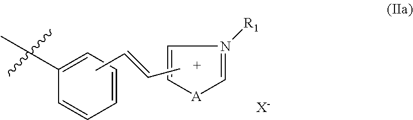

in which:
R₁ denotes a hydrogen atom or a C₁-C₄ alkyl or C₁-C₄ hydroxyalkyl group;
A denotes a sulfur atom, an oxygen atom or an NR′ or C(R′)₂ group, R′; where R′ represents a hydrogen atom or a C₁-C₄ alkyl group, and R′ preferably represents a hydrogen atom; and
X⁻ has the same meaning as that described for the preceding formula (Ia);
where

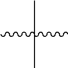

has the same meaning as previously;

preferably the styryl group is found on the para of the phenyl group

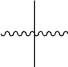

Such chemical groups bear activated double bonds, and as such the photo-dimerization of these double bonds starts spontaneously in the UVA range, without requiring a photoinitiator.

Photoinitiator is understood in the sense of the present invention as a compound that initiates the photo-dimerization reaction and releases a radical when irradiated, notably in the UV domain.

According to one particular embodiment, the composition of the invention is free of photoinitiators.

According to a particular embodiment of the invention the polymer may include one or more pendant hydrophobic groups.

As pendant hydrophobic group, mention may be made of:
saturated or unsaturated (C₁-C₃₀)alkyl groups, optionally substituted and/or interrupted with one or more heteroatoms,
alkenyl groups,
aryl groups such as phenyl, pyridyl, furyl, indolyl, benzofuryl, thiophenyl, imizadolyl, oxazolyl, thiazolyl, pyrazinyl, pyrimidinyl;
fluorinated groups such as fluorocarbon groups such as —CF₃, —CHF₂, —OCF₃, —SCF₃, CF₃C(O)—,
silicone groups such as —SiRₐR_{b}R_{c} such as —Si(CH₃)₃, polydimethylsiloxane-PDMS, —Si(OR)₃, α,ω-diaminopropyl PDMS, α,ω-dihydroxyalkyl PDMS, α,ω-dicarboxyalkyl PDMS, where Rₐ, R_{b} and R_{c}, identical or different, represent a (C₁-C₈)alkyl group optionally interrupted and/or terminated with one or more non-contiguous heteroatoms such as O or S; and R represents a (C₁-C₆)alkyl group.

Preferably the pendant hydrophobic group(s) are chosen from a (C₂-C₂₂)alkyl group, more preferentially a (C₃-C₁₆)alkyl group and notably propyl.

The polymer backbone may be of varied nature. This polymer backbone may be natural or synthetic. As natural polymer backbones, mention may be made of polysaccharides.

As polysaccharides, mention may be made of xanthan, carrageenan, chitosan, cellulose and its derivatives, alginate, starch, dextran, pullulan, galactomannan and the biologically acceptable salts thereof, and derivatives thereof. As synthetic backbones, mention may be made of poly(vinyl) polymers and polydiorganosiloxanes.

Among poly(vinyl) polymers, mention may be made of partially or totally hydrolyzed polyvinyl acetate, and of polyvinyl alcohol (PVA).

As regards the polymers containing photo-dimerizable groups bearing a stilbazolium function, they are obtained by reacting the polymer under consideration with a chemical species including a group having formula (Ia) or (Ib).

Preferably, the chemical species including a group (Ia) bears a reactive group W of aldehyde or acetal type.

As chemical species that can be used to graft styrylpyridinium groups, mention may notably be made of quaternary salts of 2-(4-formylstyryl)-pyridinium, 4-(4-formyl-styryl)-pyridinium, 2-(3-formylstyryl)-pyridinium, N-methyl-2-(4-formylstyryl)pyridinium, N-methyl-3-(4-formylstyryl)-pyridinium, N-methyl-2-(3-formylstyryl)-pyridinium, N-methyl-2-(2-formylstyryl)pyridinium, N-ethyl-2-(4-formyl-styryl)-pyridinium, N-(2-hydroxyethyl)-2-(4-formylstyryl)-pyridinium, N-(2-hydroxyethyl)-4-(4-formylstyryl)-pyridinium, N-methyl-4-(4-formylstyryl)-pyridinium, N-methyl-4-(3-formylstyryl)-pyridinium.

The pyridinium quaternary salts may be chloride, bromide, iodide, perchlorate, tetrafluoroborate, methosulfate, phosphate, sulfate, methanesulfonate or p-toluenesulfonate salts. Such chemical species are described in GB-A-2030575.

Examples of species that may be mentioned include 4-(4-formylphenylethenyl)-1-methylpyridinium methosulfate, 1-(3-ethoxycarbonylmethyl)-4-[2-(4-formylphenyl)ethenyl]pyridinium bromide and 1-(methoxycarbonylpropyl)-4-[2-(4-formylphenyl)ethenyl]pyridinium bromide. Such species are described in US 2007/0 112 094.

Use is preferably made of n-methyl-4-(4-formylstyryl)pyridinium methyl sulfate (RN=74401-04-0), sold notably by the company Wako.

These polymers functionalized with photo-dimerizable groups such as those comprising a styryl group and hydrophobic groups, can be synthesized, as described below, on the basis of the protocol of T. Uhlich et al. (Reactive & Functional Polymers, 28, 55-40 (1995)).

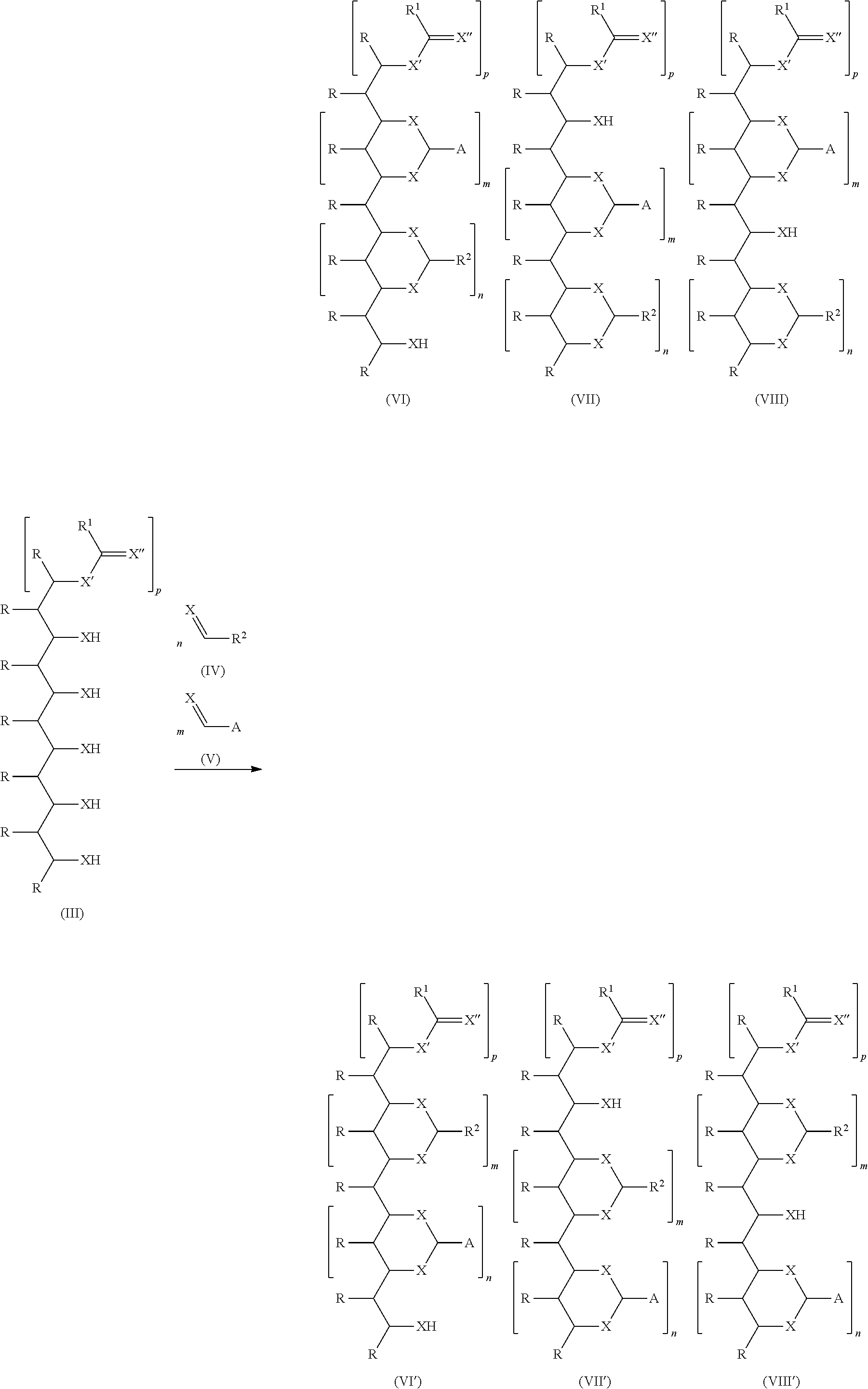

Compound having formula (III) to (VIII′) in which,

R, which may be identical or different, represents a hydrogen atom, or a (C₁-C₁₀)alkyl group, optionally substituted and/or interrupted with one or more heteroatoms, preferably R represents a hydrogen atom or a (C₁-C₄)alkyl group such as methyl, ethyl, or propyl, more preferentially R represents a hydrogen atom;

R¹ represents a hydrogen atom, or a (C₁-C₁₀)alkyl group, optionally substituted and/or interrupted with one or more heteroatoms, preferably R¹ represents a hydrogen atom or a (C₁-C₄)alkyl group;

R² represents a saturated or unsaturated (C₁-C₃₀)alkyl group, optionally substituted and/or interrupted with one or more heteroatoms, an alkenyl group, an aryl group such as phenyl, pyridyl, furyl, indolyl, benzofuryl, thiophenyl, imizadolyl, oxazolyl, thiazolyl, pyrazinyl, pyrimidinyl; a fluorinated group such as a fluorocarbon group such as —CF₃, —CHF₂, —OCF₃, —SCF₃, CF₃C(O)—, a silicone group such as —SiRₐR_{b}R_{c} such as —Si(CH₃)₃, polydimethylsiloxane-PDMS, —Si(OR)₃, α,ω-diaminopropyl PDMS, α,ω-dihydroxyalkyl PDMS, α,ω-dicarboxyalkyl PDMS, with Rₐ, R_{b} and R_{c}, which may be identical or different, representing a (C₁-C₈)alkyl group optionally interrupted and/or terminated with one or more non-contiguous heteroatoms such as 0, or S; and where R represents a (C₁-C₆)alkyl group; preferably R² represents a (C₂-C₂₂)alkyl group, more preferentially (C₃-C₁₆)alkyl;

A represents a group derived from a photo-dimerizable compound, preferably styrylpyridinium such as (I), (II), (A₁), (A₂), (Ia), (Ib) or (IIa) as defined previously, more particularly chosen from (A₁) or (Ia) as defined previously;

X represents an oxygen or sulfur, preferably oxygen, atom;

X′, and X″ represent an oxygen or sulfur atom or an N(R³) group with R³ denoting a hydrogen atom or a (C₁-C₄)alkyl group; preferably, X and X″ represent an oxygen atom;

Mainly the products obtained have formula (VI).

Advantageously, these chemical species react with a polyvinyl alcohol or polyvinyl acetal type polymer as described in the documents cited previously and also such as polymer (III) described in the scheme above for which X, X and X″ represent an oxygen atom, R and R¹ being as described previously.

For example, a grafted polyvinyl alcohol polymer including the following structural units results, where A represents a group (I), (A₁) or (Ia):

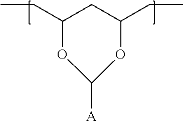

Polyvinyl alcohol polymers grafted with a styrylpyridinium group are notably described in the publication Ichimura K. et al., Preparation and characteristics of photo-crosslinkable poly(vinyl alcohol), Journal of Polymer Science, Polymer Chemistry Edition, Vol. 20, 1419-1432 (1982).

The polymers may be obtained by reacting polyvinyl alcohol or partially hydrolyzed polyvinyl acetate with styrylpyridinium salts bearing a formyl or acetal group as described in GB-A-2 030 575, WO 96/29312, U.S. Pat. No. 5,061,603, GB-A-2 076 826 and EP-A-092 901.

Cellulose polymers grafted with styrylpyridinium groups are notably described in US 2007/0 112 094.

Preferably, the chemical species including a group (A₁) or (Ia) bears one reactive group that is a halogen atom such as chlorine.

In this variant, the chemical species corresponds for example to the formula:

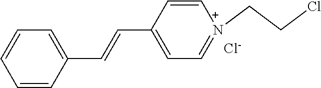 [SMILES: ClCC[n+]1ccc(/C=C/c2ccccc2)cc1 ; [Cl-]]

Advantageously, the photo-crosslinkable polymer comprising groups (Ib) is, for example, obtained by reacting the above species with the polysaccharide chosen from those defined previously.

As regards the polymers containing photo-dimerizable groups bearing a styrylazolium function, they are obtained by reacting the polymer with a chemical species comprising a group having formula (IIa).

Preferably, the chemical species including a group (IIa) bears a reactive group W of aldehyde or acetal type.

As chemical species that may be used to graft groups of styrylazolium type, mention may be made of those described in EP-A-313 220.

Advantageously, these chemical species react with a polymer of polyvinyl alcohol or polyvinyl acetate type as described in the documents cited previously.

A grafted polyvinyl alcohol polymer including the following structural units results

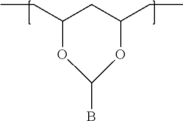

with B corresponding to the group

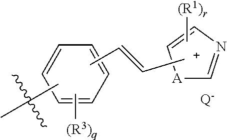

or (IIa) as defined previously.

Polyvinyl alcohol polymers grafted with styrylazolium groups are described notably in EP-A-313 220. In said document, these polymers may be obtained by reaction of polyvinyl alcohol or partially hydrolyzed polyvinyl acetate with styrylazolium salts bearing an aldehyde or acetal group.

According to one embodiment, the polymer bearing photo-dimerizable group(s) are in the form of particles, in particular of dispersed particles. Thus, in this latter case, the polymer particles are very preferentially polyvinyl alcohol particles.

According to a preferred embodiment, the polymer bearing photo-dimerizable group(s) of the invention is soluble in the cosmetic medium.

Thus, according to one embodiment variant, the polymer is a polyvinyl alcohol (PVA) polymer partly functionalized with one or more hydroxyl functions and one or more functions having formula (IX):

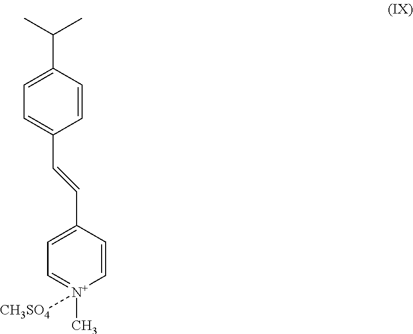

The degree of polymerization of the PVA may be between 100 and 5000 and the level of substitution, in % of functions having formula (I) as defined above, may be between 0.1 and 25.

The following scheme represents one variant where the polymer is the polymer (III) as defined previously bearing functions grafted with stylbazolium species such as those having formula (A1) as defined previously, which can crosslink under the effect of light, as illustrated below.

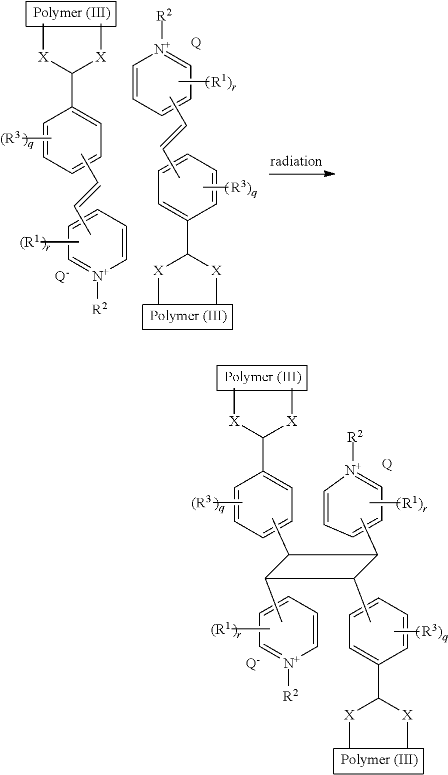

These materials react to radiation that may include both a UV light component and a visible light component, particularly a low dose of UV.

Preferentially, the following scheme represents the polymer which is PVA-SbQ (polymer of polyvinyl acetate type bearing a few hydrolyzed functions and a few functions grafted with stilbazolium species), which can crosslink under the effect of light, as illustrated below.

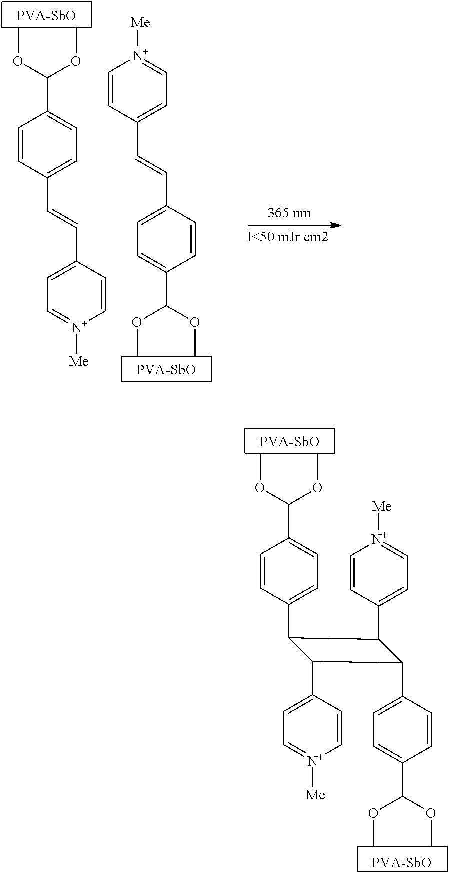 [SMILES: COC(OC)c1ccc(/C=C/c2cc[n+](C)cc2)cc1 ; COC(OC)c1ccc(/C=C/c2cc[n+](C)cc2)cc1 ; COC(OC)c1ccc(C2C(c3cc[n+](C)cc3)C(c3ccc(C(OC)OC)cc3)C2c2cc[n+](C)cc2)cc1]

These materials are particularly appreciated since they do not require a photoinitiator and react with radiation that may include both UV light and visible light, in particular a low dose of UV.

Pendants groups which are reactive in both UV light and visible light are preferred.

According to another embodiment variant, the photo-crosslinkable polymer is featured by a natural polymer that is functionalized with photo-dimerizable groups.

It may notably be a polysaccharide that may notably be chosen from chondroitin sulfate, keratan, keratan sulfate, heparin, heparin sulfate, xanthan, carrageenan, hyaluronic acid, chitosan, cellulose and derivatives thereof, alginate, starch, dextran, pullulan, galactomannan and biologically acceptable salts thereof.

The degree of functionalization is of course adjusted be able to provide the degree of crosslinking required during activation.

According to the invention, the degree of functionalization with photo-dimerizable units is at least 0.1%, or even at least 0.5%, or even at least 2%.

Preferably, in a composition according to the invention, the photo-dimerizable groups are borne by a polyvinyl acetate or polysaccharide polymer.

The crosslinkable polymer may be conveyed in an aqueous medium.

The composition may contain a single polymer bearing photo-dimerizable pendant groups that may or may not be of different nature.

Use may also be made of a mixture of polymers having different functions.

Consequently, the reactions may take place between two photo-dimerizable groups that may or may not be of the same chemical nature.

The activated double bonds may react with another double bond of the same chemical nature or may react with another double bond of different chemical nature.

As examples of polymers that are useful in the invention, mention may be made of the polymer PVA comprising the pendant groups below called PVA-SbQ-propional in which, preferably, the quantity of SbQ units is inclusively between 0.5 and 5 mol %, preferably between 2 and 4 mol %, for example of the order of 2 mol %, the quantity of propional groups is inclusively between 2 and 20 mol %, preferably between 5 and 15 mol %, for example of the order of 10 mol %, the quantity of hydroxyl groups is inclusively between 50 and 97.5 mol %, preferably between 60 and 97.5 mol %, for example about 86 mol %:

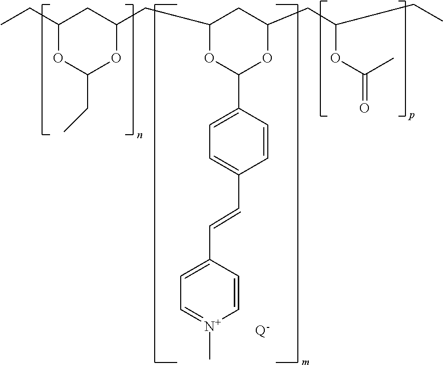

with Q⁻ as defined previously, preferably mesylate CH₃OSO₃⁻.

According to one particular embodiment, the molecular weight Mw of the PVA is between 10 000 and 100 000 g/mol and preferably between 25 000 and 80 000 g/mol.

According to one particular embodiment, the molecular weight Mw is of the order of 27 000 g/mol.

The polymer(s) including at least one photo-dimerizable group preferably represent 0.01 to 25%, better still 0.1 to 20%, and even better still 1 to 15% relative to the total weight of the composition.

The composition according to the invention may also comprise an effective quantity of at least one photosensitizing agent.

In the sense of the present invention, photosensitizing agent is understood to mean an ingredient that modifies the irradiation wavelength, thereby triggering the photo-dimerization reaction.

For example, the photo-dimerization of dimethylmaleimide groups is triggered by irradiation centered on the wavelength range from 270 to 300 nm. In the presence of a photosensitizing agent such as thioxanthone, photo-dimerization becomes effective with irradiation centered on the wavelength domain ranging from 360 to 430 nm.

Among the photosensitizers that can be used according to the invention, mention may notably be made of thioxanthone, rose Bengal, phloxine, eosin, erythrosine, fluorescein, acriflavine, thionine, riboflavin, proflavine, chlorophylls, hematoporphyrin, methylene blue and mixtures thereof.

In practice, the photosensitizing agent that can be used according to the invention represents 0.00001% to 5% of the total weight of the composition.

The composition according to the invention also comprises one or more alkaline agent(s) and/or one or amino alkoxysilane derivative(s).

The alkaline agent(s) may be chosen from:
water-soluble mineral alkaline agents such as
   hydroxides of alkali metals or alkaline-earth metals, preferably of lithium, magnesium, calcium, sodium or potassium buffered with one or amino acids such as glycine,
   carbonates, bicarbonates or hydrogen carbonates, carbonates buffered with bicarbonate to obtain a pH<12.0,
   phosphates of alkali metals or alkaline-earth metals such as lithium, calcium, sodium or potassium,
aqueous ammonia,
   alkanolamines such as monoethanolamine, diethanolamine and triethanolamine, mono-, di- and tri-(hydroxymethyl)aminomethane and also derivatives thereof,
basic amino acids,
alkaline polyamino acids or alkaline amino polyacids, such as polylysines and polyarginines,
polyamines such as polyethyleneimines (PEI), polyvinylamines and the compounds of formula (A) below:

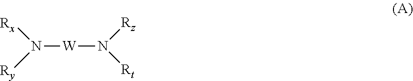

in which formula (A) W is a (C₁-C₆)alkylene group such as propylene optionally substituted with a hydroxyl or amino group or a C₁-C₄ alkyl radical; Rₓ, R_{y}, R_{z} and Rₜ, which may be identical or different, represent a hydrogen atom or a C₁-C₄ alkyl or C₁-C₄ hydroxyalkyl radical.

Examples of amines of formula (A) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁-C₈ alkyl groups bearing one or more hydroxyl radicals.

Organic amines chosen from alkanolamines such as mono-, di- or tri-alkanolamines comprising from 1 to 3 identical or different C₁-C₄ hydroxyalkyl radicals are particularly suitable for performing the invention.

Among compounds of this type, mention may be made of monoethanolamine (MEA), diethanolamine, triethanolamine, monoisopropanolamine, diisopropanolamine, N,N-dimethylethanolamine, aminopropanol, 1-aminopropanol, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane, aminobutanol, aminopentanol and aminohexanol, preferably 2-amino-2-methyl-1-propanol.

More particularly, the amino acids that may be used are of natural or synthetic origin, in their L, D or racemic form and include at least one acid function chosen more particularly from carboxylic, sulfonic, phosphonic or phosphoric acid functions.

As amino acids that may be used in the present invention, mention may notably be made of basic amino acids comprising an additional amine function optionally included in a ring or in a ureido function.

Such basic amino acids are preferably chosen from those corresponding to formula (B) below, and also the salts thereof

R—CH2-CH(NH2)-C(O)—OH (B)

in which formula (B) R represents a group chosen from: imidazolyl, preferably 5-imidazolyl; —(CH₂)₃—NH₂; —(CH₂)₂—NH₂; —(CH₂)₂N(H)—C(O)—NH₂; and —(CH₂)₂—N(H)—C(NH)—NH₂.

The compounds corresponding to formula (B) are histidine, lysine, arginine, ornithine and citrulline.

The organic amine may also be chosen from organic amines of heterocyclic type. Mention may be made in particular, besides histidine that has already been mentioned among the amino acids, of pyridine, piperidine, imidazole, triazole, tetrazole and benzimidazole.

The term “poly” amines or “poly” amino acids means alkaline agents comprising at least two amino or amino acid groups connected to the same molecule; they are in particular polymers whose backbone comprises or is substituted with one or more amine groups or amino acid groups.

The organic amine may also be chosen from amino acid dipeptides. As amino acid dipeptides that may be used in the present invention, mention may notably be made of carnosine, anserine and whale.

The organic amine may also be chosen from compounds including a guanidine function. As amines of this type that may be used in the present invention, mention may notably be made, besides arginine that has already been mentioned as an amino acid, of creatine, creatinine, 1,1-dimethylguanidine, 1,1-diethylguanidine, glycocyamine, metformin, agmatine, N-amidinoalanine, 3-guanidinopropionic acid, 4-guanidinobutyric acid and 2-([amino(imino)methyl]amino)ethane-1-sulfonic acid.

As hybrid compounds, mention may be made of salts of the amines mentioned previously with acids such as carbonic acid or hydrochloric acid.

Use may be made in particular of guanidine carbonate or monoethanolamine hydrochloride.

Preferably, the alkaline agent(s) are chosen from water-soluble mineral alkaline agents, alkanolamines, basic amino acids and organic amines, preferably organic amines.

Preferably, the alkaline agent(s) are chosen from hydroxides of alkali metals or alkaline-earth metals, preferably of lithium, magnesium, calcium, sodium or potassium buffered with one or more amino acids such as glycine, carbonates, bicarbonates or hydrogen carbonates, carbonates buffered with bicarbonate to obtain a pH<12.0, phosphates of alkali metals or alkaline-earth metals such as lithium, calcium, sodium or potassium, monoethanolamine (MEA), aminopropanol, 1-aminopropanol, 2-amino-2-methyl-1-propanol, 2-amino-2-methyl-1,3-propanediol, aminobutanol, aminopentanol, aminohexanol, arginine and guanidine.

The amino alkoxysilane derivative(s) may be chosen from the compounds corresponding to formula (C) below and/or oligomers thereof and/or hydrolysis products thereof:

R₁Si(OR₂)_{z}(R₃)ₓ(OH)_{y} (C)

in which formula (II):
R₁ is a linear or branched, saturated or unsaturated, cyclic or acyclic C₁-C₆ hydrocarbon-based chain substituted with a group chosen from the following groups:
amine NH₂ or NHR (R═C₁-C₂₀ and notably C₁-C₆ alkyl optionally substituted with a radical including a silicon atom, C₃-C₄₀ cycloalkyl or C₆-C₃₀ aromatic),
an aryl or aryloxy group substituted and/or interrupted with at least one amino group or with at least one C₁-C₄ aminoalkyl group,

R₁ possibly being interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2,

with z+x+y=3.

The term “oligomer” means products of polymerization of the compounds of formula (C) including from 2 to 10 silicon atoms.

Preferably, R₂ represents an alkyl group comprising from 1 to 4 carbon atoms, better still a linear alkyl group comprising from 1 to 4 carbon atoms, and preferably an ethyl group.

Preferably, R₃ represents an alkyl group comprising from 1 to 4 carbon atoms, better still a linear alkyl group comprising from 1 to 4 carbon atoms, and preferably methyl or ethyl groups.

Preferably, R₁ is an acyclic chain.

Preferably, the compound of formula (C) includes only one silicon atom in its structure.

Preferably, R₁ represents an alkyl group, and even more preferentially a linear alkyl group, comprising from 1 to 6 carbon atoms or a C₁-C₆ aminoalkyl group.

Preferably, z ranges from 1 to 3. Even more preferentially, z is equal to 3.

Preferably, R₁ is a linear or branched, saturated or unsaturated C₁-C₆ hydrocarbon-based chain substituted with an amine group NH₂ or NHR (R═C₁-C₂₀ and notably C₁-C₆ alkyl, C₃-C₄₀ cycloalkyl or C₆-C₃₀ aromatic).

Preferably, the first composition comprises at least one compound of formula (C) chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane, better still from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane, or oligomers thereof and/or hydrolysis products thereof.

According to a particularly preferred embodiment, the composition comprises 3-aminopropyltriethoxysilane (APTES) or oligomers thereof and/or hydrolysis products thereof.

According to the invention, the alkaline agent(s) and/or the amino alkoxysilane derivative(s) used is/are in the composition in a content ranging from 0.01% to 10% by weight, preferentially between 0.02% and 5% by weight relative to the total weight of the composition.

The pH of the composition according to the invention is greater than or equal to 7.

Preferably, the composition of the invention has a pH ranging from 7.0 to 12.0, preferentially from 7.5 to 11, better still from 8 to 10 and even more preferentially from 8 to 9.

Preferably, the pH of the composition is stable, i.e. it remains basic, that is to say greater than or equal to 7, over time. Preferably, the pH of the composition remains basic for a storage time of at least 4 weeks, or even 8 weeks.

The composition may comprise a cosmetically acceptable medium. The cosmetically acceptable medium that may be used in the compositions of the invention may comprise a solvent chosen from water, organic solvents, and a mixture thereof.

The organic solvents may be chosen from alcohols, polyols, polyol ethers and mixtures thereof, the alcohols being preferentially chosen from lower C₁-C₆ alkanols, and preferably chosen from ethanol, propanol and isopropanol, the polyols being preferentially chosen from propylene glycol, hexylene glycol, glycerine, and pentanediol.

Preferably, the solvent(s) represent 0.1% to 99% of the total weight of the composition.

The composition is preferably aqueous.

According to this variant, the amount of water may range from 5 to 98% by weight, better from 15% by weight to 95% by weight, better still from 25 to 90% by weight and even more preferentially from 30 to 90% by weight, relative to the total weight of the composition.

The composition according to the invention may also contain additives usually used in cosmetics, such as anionic, cationic, amphoteric or nonionic surfactants, thickeners, fatty substances, fixing or conditioning polymers, preserving agents, fragrances, colorants, pigments, oxidizing agents, antidandruff agents, agents for preventing hair loss, agents for breaking disulfide bonds, and also most of the usual cosmetic agents.

These additives may be present in the composition according to the invention in an amount ranging from 0 to 20% by weight, relative to the total weight of the composition.

A person skilled in the art will take care to select these optional additives and amounts thereof so that they do not harm the properties of the compositions of the present invention.

The process of the invention comprises, after step a) of applying the composition, a step b) of irradiating the composition on the keratin materials to crosslink the polymer.

This irradiation may consist of illumination, with ambient light or with a source of artificial light, of the composition applied to the keratin materials.

The ambient or artificial light may emit radiation in the visible and/or UV range. Preferably, it emits at least a proportion of radiation in the UV range, for example a UV proportion of at least 2% of the total illuminating energy of the ambient light.

According to a particular embodiment, the exposure comprises, or even consists of, illumination with ambient light of the surface of said coat, in particular for a time of at least 1 minute.

The exposure time to the ambient light may range more particularly from 10 seconds to 30 minutes and notably from 2 to 15 minutes.

According to another particular embodiment, the exposure comprises, or even consists of, illumination with a source of artificial light of the surface of said coat.

The exposure time to said artificial light may range from 1 second to 20 minutes and in particular from 1 second to 1 minute.

The crosslinking may take place with natural or artificial light, for example using lighting with a lamp, a flash, a laser or LEDs, for example in the form of an LED array.

The artificial light source may emit radiation in the visible range and/or radiation in the UV range.

The light emitted may or may not be monochromatic. The wavelength of the emitted light is preferably centered on 365 nm, in particular between 100 nm and 500 nm and better still between 200 nm and 420 nm.

Advantageously, the crosslinking is initiated by simple illumination without the need for a photoinitiator.

Preferably, it will be a source of artificial light emitting energy between 0.5 and 5 W/cm², the exposure times being adapted in consequence.

The crosslinking may occur with reduced light intensity, the lighting system may produce this light intensity for example between 500 mJ/cm² and 10 J/cm².

The twofold characteristic of the absence of a photoinitiator and the relatively low light intensity is particularly advantageous since it makes it possible to limit the harmful effects of aggressive initiators or of prolonged exposure to intense light, in particular in the UV wavelengths.

A person skilled in the art will be capable of adapting the illumination characteristics, notably in terms of exposure time and of radiation wavelengths, with regard to the nature of the photo-crosslinkable polymer(s) (A) used.

According to a preferred embodiment, the composition is applied to keratin fibers such as hair.

According to this embodiment, the composition may be applied to wet or dry, clean or unclean keratin fibers. Preferably, the keratin fibers are dried after applying the composition and before irradiation.

In the process, before or after irradiation step b), a pause at room temperature, or high temperature, or under red light, may also be included in the process.

The example below illustrates the invention without, however, limiting the scope thereof. The quantities are given, unless otherwise indicated, in percentages by weight.

### EXAMPLE

The following compositions were prepared from the ingredients of which the contents are indicated in the table below (% in g of active material).

TABLE 1

| | 1 | 2 | 3 | 4 | 5 |
|---|---|---|---|---|---|
| PVA-SbQ* | 10 | 10 | 10 | — | — |
| PVA-SbQ-propional** | — | — | — | 10 | 14.9 |
| NaOH | 3 | — | — | 3 | |
| Aminomethylpropanol | — | 1.3 | — | | 5 |
| Monoethanolamine | — | — | 0.03 | — | — |
| Water QS | 100 | 100 | 100 | 100 | 100 |
| pH | 8.4 | 8.8 | 8.6 | 8 | 8.5 |

*partially saponified polymer of polyvinyl acetate type bearing stilbazolium groups, adsorbed on polyvinyl acetate particles
**PVA-SbQ-Propional obtained according to the process described previously
Compositions which have a pH that is stable over time are obtained, affording the polymer stability.

Uniform and persistent coating was obtained on the hair.

Results of the same type were obtained for PVA-SbQ bearing phenyl groups.

1.-22. (canceled)

23. A composition comprising:
at least one photo-crosslinkable polymer comprising at least one photo-dimerizable pendant group; and
at least one alkaline agent and/or amino alkoxysilane derivative present in an amount ranging from 0.01% to 10% by weight relative to the total weight of the composition,
wherein the pH of the composition is greater than or equal to 7.

24. The composition of claim 23, wherein the at least one photo-dimerizable group is chosen from monovalent radicals having the formula (I) or (II): 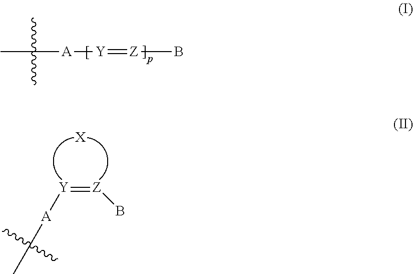
or geometric isomers thereof,
wherein:
Y and Z independently represent a nitrogen atom or a group C(R), wherein R represents a hydrogen atom or a (C₁-C₄)alkyl group;
A represents one bond or a divalent group chosen from (C₁-C₈)alkylene, arylene, heteroarylene, cycloalkylene, heterocycloalkylene, (thio)carbonyl, (C₂-C₈)alkenylene radicals or combinations thereof;
B represents a monovalent group chosen from (C₁-C₈)alkyl radicals, aryl, or combinations thereof;
X represents a divalent group chosen from (C₂-C₈)alkylene, arylene, heteroarylene, cycloalkylene, heterocycloalkylene, (thio)carbonyl, (C₂-C₈)alkenylene radicals or combinations thereof;
p represents an integer between 1 and 5 inclusive; 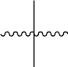
represents the bond that connects the part of the monovalent radical to the rest of the molecule;
wherein each of the groups cited can optionally be substituted with one or more halogen atoms or groups chosen from (C₁-C₆)alkyl, hydroxy, amino, (di)(C₁-C₆)alkylamino, phenyl, carboxy, (C₁-C₆)alkoxy, (C₁-C₆)alkoxy(thio)carbonyl, hydrogen(thio)-carbonyl, sulfonato R—O—S(O)₂— or R—S(O)₂—O—, amide RR′N—C(O)— or R—C(O)—N(R′)— or acyl R—C(O)—, ammonium RR′R″N⁺— groups, wherein R, R′, and R″, which may be identical or different, represent a hydrogen atom or a (C₁-C₄)alkyl group.

25. The composition of claim 24, wherein the at least one photo-dimerizable group is chosen from monovalent radicals of the following compounds:
a) stilbene,
b) styrylpyridinium (stilbazolium) having formula (A₁) or (A₂): 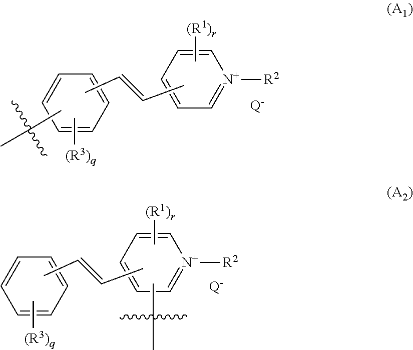
or geometric isomers thereof,
wherein:
R¹ and R³, which may be identical or different, represent a halogen atom or a (C₁-C₆)alkyl group, or two contiguous R¹ or R³ groups that together form a benzo group with the carbon atoms that bear them;
R² represents a hydrogen atom, a (C₁-C₆)alkyl group optionally substituted with one or more halogen atoms;
q and r represent an integer ranging from 0 to 4;
Q⁻ represents an anionic counterion; and 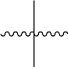
represents a bond connecting the monovalent radical to the rest of the molecule, wherein the pendant group A2 is optionally connected to the rest of the molecule via R², wherein the 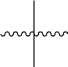
bond is found on the phenyl in the para position of the styryl group on A₁ or connected to the rest of the molecule via R² on A2;
c) styrylazolium having formula (B1) or (B2) or geometric isomers thereof: 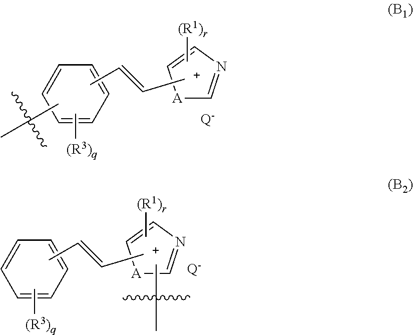
wherein:
A represents a sulfur atom, an oxygen atom, or an NR² or C(R²)₂ group; wherein 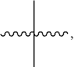
Q⁻, r, q, R¹, R², and R³ are as defined previously,
d) styrylpyrazine,
e) chalcone,
f) (thio)cinnamate or (thio)cinnamamide,
g) maleimide,
h) (thio)coumarin,
i) thymine,
j) uracil,
k) butadiene
l) anthracene,
m) pyridone,
n) pyrrolizinone,
o) acridizinium salts,
p) furanone,
q) phenylbenzoxazole, or
r) derivatives thereof.

26. The composition of claim 23, wherein the at least one photo-crosslinkable polymer has a natural or a synthetic backbone chosen from polysaccharides, poly(vinyl) polymers, or polydiorganosiloxanes.

27. The composition of claim 23, wherein the at least one photo-crosslinkable polymer is soluble or dispersed in the composition.

28. The composition of claim 23, wherein the at least one photo-crosslinkable polymer is in the form of particles.

29. The composition of claim 28, wherein the particles are polyvinyl alcohol particles.

30. The composition of claim 23, wherein the at least one photo-crosslinkable polymer includes at least one hydrophobic pendant group.

31. The composition of claim 30, wherein the hydrophobic group is chosen from:
a saturated or unsaturated (C₁-C₃₀)alkyl group,
an alkenyl group,
an aryl group,
a fluorinated group, or
a silicone group.

32. The composition of claim 23, wherein the composition comprises the at least one photo-crosslinkable polymer in an amount ranging from 0.01 to 25% relative to the total weight of the composition.

33. The composition of claim 23, wherein the at least one alkaline agent is chosen from aqueous ammonia, water-soluble mineral alkaline agents or alkaline-earth metals, carbonates or phosphates of alkali metals or alkaline-earth metals, alkanolamines or derivatives thereof, basic amino acids, alkaline polyamino acids, polyamines, polyvinylamines, or compounds of formula (A) below: 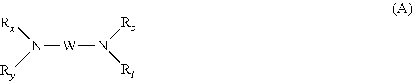
wherein W is a (C₁-C₆)alkylene group, and Rₓ, R_{y}, R_{z} and Rₜ, which may be identical or different, represent a hydrogen atom, a C₁-C₄ alkyl, or a C₁-C₄ hydroxyalkyl radical.

34. The composition of claim 23, wherein the at least one alkaline agent is chosen from water-soluble mineral alkaline agents, alkanolamines, basic amino acids, or organic amines.

35. The composition of claim 23, wherein the at least one alkaline agent is chosen from hydroxides of alkali metals or alkaline-earth metals.

36. The composition of claim 23, wherein the at least one amino alkoxysilane derivative corresponds to formula (C) below and/or oligomers thereof:
R₁Si(OR₂)_{z}(R₃)ₓ(OH)_{y} (C)
wherein:
a) R₁ is a linear or branched, saturated or unsaturated, cyclic or acyclic C₁-C₆ hydrocarbon-based chain substituted with a group chosen from the following groups:
amine NH₂ or NHR, wherein R is a C₁-C₂₀ alkyl optionally substituted with a radical including a silicon atom, a C₃-C₄₀ cycloalkyl or a C₆-C₃₀ aromatic, or
an aryl or aryloxy group substituted and/or interrupted with at least one amino group or with at least one C₁-C₄ aminoalkyl group, and
wherein R₁ is optionally interrupted with O, S, NH, or CO,
b) R₂ and R₃, which may be identical or different, represent a linear or branched alkyl group comprising from 1 to 6 carbon atoms,
c) y represents an integer ranging from 0 to 3,
d) z represents an integer ranging from 0 to 3, and
e) x represents an integer ranging from 0 to 2,
wherein z+x+y=3.

37. The composition of claim 23, further comprising at least one compound chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane N-(2-aminoethylaminomethyl)phenethyltrimethoxy-silane, or oligomers and/or hydrolysis products thereof.

38. The composition of claim 23, further comprising at least one compound chosen from 3-aminopropyltriethoxysilane or oligomers and/or hydrolysis products thereof.

39. The composition of claim 23, wherein the composition comprises at least one alkaline agent and/or amino alkoxysilane derivative in an amount ranging from 0.01% to 10% by weight relative to the total weight of the composition.

40. The composition of claim 23, wherein the pH of the composition ranges from 7.5 to 11.

41. A process for treating keratin materials comprising the following steps:
a) application of a composition to the keratin materials, the composition comprising:
at least one photo-crosslinkable polymer comprising at least one photo-dimerizable pendant group; and
at least one alkaline agent and/or amino alkoxysilane derivative present in the composition in a content ranging from 0.01% to 10% by weight relative to the total weight of the composition,
wherein the pH of the composition is greater than or equal to 7;
b) irradiation of said composition on the keratin materials to crosslink the polymer.

42. The process of claim 41, wherein
a) the keratin materials are dried after application of the composition to the keratin materials and before the irradiation of the composition,
b) the keratin materials are exposed to red light at room temperature or higher after application of the composition to the keratin materials and before the irradiation of the composition, and/or
c) the irradiation is irradiation with radiation in the UVA or the visible domain.
